(19) United States
(12) Patent Application Publication
(10) Pub. No.: US 2024/0360481 A1
(43) Pub. Date: Oct. 31, 2024

(54) BIOCOMPATIBLE UNDERLAYS FOR LIVING EXTRACTION OF HYDROCARBONS FROM ENGINEERED MICROBES

(71) Applicant: King Abdullah University of Science and Technology, Thuwal (SA)

(72) Inventors: Kyle Jonathan Lauersen, Thuwal (SA); Sebastian Overman, Thuwal (SA)

(21) Appl. No.: 18/688,207

(22) PCT Filed: Aug. 30, 2022
(86) PCT No.: PCT/IB2022/058125

§ 371 Date: Feb. 29, 2024

Related U.S. Application Data
(60) Provisional application No. 63/239,142, filed on Aug. 31, 2021.

(51) Int. Cl. C12P 5/00; C12M 1/00; C12N 1/12; C12R 1/89

(52) U.S. Cl. CPC C12P 5/007; C12M 23/40; C12M 43/00; C12N 1/12; C12R 2001/89

(57) ABSTRACT

Methods and systems that use fluorocarbons for designing scalable living, in-line extraction of heterologous metabolites from microbial cultures are disclosed. The disclosed systems and methods are organism agnostic and be applied to bacteria, yeasts, filamentous microbes, and algae. The disclosed methods and systems use pperfluorocarbon solvent to extract terpenes from engineered microorganisms and allows non-lethal milking of hydrocarbon products from filamentous and single-celled/multicelled microbial cultures. The disclosed process preferably does not use pressure gradients and relies on the physical interaction of microbes with fluorocarbon at satp and standard temperature of microbial cultivation to partition non-native products. The disclosed process and system also allow for increased production of products providing a physical sink which enables the forward reactions to produce more products than in its absence.

### CROSS-REFERENCE TO RELATED APPLICATION

The present application claims priority to U.S. Application No. 63/239,142 filed Aug. 31, 2021, the disclosure of which is incorporated herein by reference in its entirety.

### FIELD OF THE INVENTION

The disclosed invention is generally in the field of extraction of chemicals (for example, terpenoid hydrocarbons) that are extractable by hydrophobic solvents from living microbial cells during cultivation and specifically in the area of using fluorinated hydrocarbons “fluorocarbons” which are liquid at standard ambient temperature and pressure (SATP) for the extraction of such chemicals from living microbial cells during cultivation.

### BACKGROUND OF THE INVENTION

Engineered microbes, including yeasts, bacteria, and more recently, algae have been used for more than a decade to produce non-native metabolites by the expression of heterologous pathways from other organisms. This process can hijack cellular metabolism and re-route it for the production of specialty chemicals (Kirby et al., Annual Review of Plant Biology, 60:335-355 (2002)). At lab-scale, extraction of these chemicals can be achieved using a biocompatible long-chain alkane such as decane, dodecane, heptadecane, or similar compounds, which are less-dense than water, sit on top of the microbial culture, and act as a physical sink which terpenoid compounds more favorably partition into than the microbial cells, or culture broth (Beekwilder, et al., Plant Biotechnology Journal, 12 (2): 174-182 (2013)). Unfortunately, having this bio-compatible overlay on top of a turbid, mixed, liquid microbial culture with gassing has limitations in that it can form emulsions with hydrophobic cell components, which limits its lifetime, and product extractability as well as reactor designs (Lauersen, Planta, 249:155-180 (2019)).

Hydrocarbon solvent-medium dual-phase concepts are convenient at small scales, however, they have significant drawbacks that prevent their use at volumes greater than a few hundred milliliters. Alkane solvents are flammable and require specialized infrastructure for pumping, as they are incompatible with silicone-based tubing. These solvents are also distillates of petroleum, which can contain many contaminants depending on the supplier. The most notable issue with using hydrophobic solvent overlays is the difficulty in scaling their application to larger culture volumes in turbid and sparged bioreactor concepts.³ As an overlay, any turbidity at the culture-solvent interface creates an emulsion of hydrophobic cellular components and the solvent that can blow out of reactors or lyse cells, hindering the processibility and productivity of extracted isoprenoids.

It is an object of the present invention to provide methods for extracting metabolites of interest that accumulate in hydrophobic solvents from microbial cultures.

It is also an object of the present invention to provide compositions for extracting metabolites of interest that accumulate in hydrophobic solvents products from microbial cultures.

It is also an object of the present invention to provide a system for extracting metabolites of interest that accumulate in hydrophobic solvents products from microbial cultures.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

Methods and systems for scalable living, in-line extraction of homologous/heterologous metabolites that can accumulate in a hydrophobic solvent, from living cell cultures such as microbial or plant cell cultures are disclosed. The microbial culture can be a static culture or a non-static (agitated) culture.

In one particularly preferred embodiment, the metabolite is an isoprenoid hydrocarbon. The methods and systems use a first extraction composition that is preferably a solvent which is denser than water, allowing for formation of an underlay in microbial cultures, are biocompatible, non-toxic and non-environmentally hazardous, and are preferably liquid at biologically relevant temperatures, thus enabling a non-destructive hydrocarbon extraction from living systems for continuous bio-production of the product of interest. The first extraction composition in one embodiment is a solvent that is preferably a substituted (with FL, Cl, Br or I) hydrocarbon. In one preferred embodiment, the solvent is a liquid perfluorocarbon, for example, FC-3283, FC 40, FC 770 or FC 3284. The process includes the steps of (1) contacting the raw material (present in a microbial culture/produced by a microbial culture)/microbial culture with a first extraction composition so as to extract the compound or composition of interest from the raw material/microbial culture into the first extraction composition and (2) separating the first extraction composition containing the extracted metabolite from the raw material. The method also includes a secondary direct extraction step (liquid-liquid/liquid-solid extraction) using a second extraction composition which can be a solid or liquid. In a preferred embodiment, the second extraction composition is an environmentally friendly chemical such as ethanol, acetone and methanol used for liquid/liquid extraction, for example.

Also disclosed are systems for extracting metabolites of interest that accumulate in hydrophobic solvents as products from microbial cultures. The disclosed systems include lab-scale and scaled up extraction of desired products from microbial cultures. The systems include: (a) microbial cell culture vessel such as a fermentation bioreactor or a biofilm bioreactor, (b) one or more extraction vessels and (c) a collection vessel. The microbial cell culture vessel includes a microbial cell population producing the metabolite of interest, and the first extraction composition as an underlay or interface to the microbial cell population. The microbial cell culture can be a liquid culture, a biofilm, microbial culture in a substrate such as a matrix, foam or microencapsulation. A portion of the culture vessel containing the first extraction solvent (more-dense than water) in some embodiments is connected to the extraction vessel by pipes allowing the flow of the first extraction composition to, and from the microbial culture vessel and the extraction vessel. At least extraction vessels include a second extraction composition, which can be solid or liquid. The second extraction composition is preferably in fluid communication with a third collection vessel; thus, the extraction vessel can include an open connection to the collection vessel, allowing for fluid flow from the extraction vessel to the collection vessel. In some embodiments, the cell culture vessel includes a gas compartment, a hydrophobic porous membrane, and a means for introducing gas into the cell culture vessel for gas exchange.

The disclosed systems and methods are organism agnostic and can be applied to any microbial system that produces metabolite of interest naturally or that is engineered to do so, such as bacteria, yeasts, filamentous microbes, plant cell culture, and algae.

In a preferred embodiment, the disclosed methods and systems use liquid perfluorocarbon as a first extraction composition, to extract terpenes from engineered microorganisms and allows non-lethal milking of hydrocarbon products from microbial culture, such as filamentous and single-celled/multi-celled microbial cultures or plant cell cultures. The disclosed method preferably does not use pressure gradients and relies on the physical interaction of microbes with first extraction solvent at standard ambient temperature and pressure (SATP) used in microbial cultivation to partition the metabolite of interest. The disclosed process and system also allow for increased production of products from the organism by providing a physical sink which enables the forward reactions to produce more products than in its absence.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions. FIG. 1A shows dodecane overlay: FIG. 1B-1C show illustrations of a lab scale systems for microbial culture with fluorocarbon underlay extraction of microbial produced hydrocarbons/specialty chemicals in a fermentation bioreactor (FIG. 1B) or a biofilm reactor (FIG. 1C). FIG. 1D-1E is a schematic showing lab scale or industrial fermentation microbial culture with fluorocarbon underlay extraction of lab scale system for microbial culture with fluorocarbon underlay extraction of microbial produced hydrocarbons/specialty chemicals using a mixed floating bath liquid extraction (FIG. 1D) or a stationary floating bath liquid extraction (FIG. 1E). FIG. 1F is a schematic showing scaled fluorocarbon use in large-scale photo-microbial cultures (algae, cyanobacteria, moss). FIG. 1G is a schematic showing fluorocarbon underlay in raceway ponds. The top panel is an aerial view raceway pond. The middle panel shows a lateral view of mixed raceway with fluorocarbon underlay. The bottom panel shows a lateral view of a biofilm liquid microbial culture. FIG. 1H-1K show a schematic of scaled fluorocarbon use in microbial cultures, with fluorocarbon underlay on hydrophobic membrane gas exchanger (FIG. 1H), fluorocarbon underlay with culture separated by membranes (FIG. 1I), fluorocarbon-matrix-medium liquid sandwich (FIG. 1J) or a fluorocarbon-floating matrix (FIG. 1K). The top panel in FIG. 1H-I shows turbulent liquid microbial culture (any reactor type), and the bottom panel shows biofilm liquid microbial culture (any reactor type). The top panel in FIG. 1J shows culture immobilized in third matrix floating between layers of liquid medium and fluorocarbon (matrix material agnostic) and the bottom panel shows culture immobilized in matrix floating between layers of liquid medium and fluorocarbon (any matrix material) with membrane gas exchange. The top panel in FIG. 1K shows culture in matrix floating on fluorocarbon (any matrix material), matrix may be the culture medium and the bottom panel shows culture in matrix floating on fluorocarbon (any matrix material), with gas exchange through membrane matrix may be the culture medium. FIG. 1L-O shows lab scale microbial culture with fluorocarbon underlay using a packed bed matrix with phase exchange (multi-scale) (FIG. 1L), Liquid-liquid drop extraction (multi-scale) (FIG. 1M) or a packed bed matrix droplet extractor (multi-scale) (FIG. 1N and FIG. 1O). FIG. 1L shows a (tidal) reactor design where the FC underlay gets regularly pumped in an out of the reactor that houses the microbial matrix. As the FC replaces the culture medium and comes into contact with the microbes, the FC can extract the product. As the FC gets pumped out again, the microbes are instead submerged in medium and can acquire their nutrients from it. FIG. 1P shows a standard liquid-liquid extraction directly with fluorocarbons and microbial culture. FIG. 1Q shows fluorocarbon-culture-medium foam. FIG. 1R and FIG. 1S show fluorocarbon-culture-microdroplet encapsulation (FIG. 1R) and fluorocarbon-medium-microdroplet encapsulation (FIG. 1S). FIG. 1T is a schematic lab scale microbial culture with fluorocarbon underlay by liquid-liquid drop extraction with integrated photobioreactor system (multi-scale).

FIG. 2A-E shows lab scale culture and dodecane emulsion.

FIG. 3. Left: C. reinhardtii culture with clear underlay of FC-3283. Right: Stirred culture of C. reinhardtii with FC 3283 underlay after 6 days cultivation.

FIG. 4A. Gas chromatography flame ionization detection (GC-FID) of FC 3283 samples after 6 days underlay of various C. reinhardtii strains engineered to produce different isoprenoids. Not only does the FC3283 have very low background signal, but the engineered products have accumulated well in this fluorocarbon. FIG. 4B. Different perfluorocarbons have different extraction behavior depending on product properties. Chemical structures of FCs and target products are shown with relative product yields from each underlay.

FIG. 5A shows representative GC-FID chromatograms of dodecane and FC-3283 samples with and without patchoulol accumulated from incubation with growing engineered algal strains. (FIG. 5B) C. reinhardtiicultures with either dodecane overlay (left) or FC-3283 underlay (right) after 6 d on a rotary shaker with second phase. (FIG. 5C) Cell densities and (FIG. 5D) Fv/Fm of C. reinhardtii cultures during a 10 day microtiter plate experiment with either a 5% (v/v) dodecane overlay or FC-3283 underlay added to the culture (n=3). (FIG. 5E) GC-FID chromatograms of FC-3283 underlays from a C. reinhardtii wild type culture and various terpenoid-producing engineered C. reinhardtii strains harvested after 6 days cultivation.

FIG. 6A shows shaken (left) and static (right) C. reinhardtii cultures with FC-3283 underlay. FIG. 6B Patchoulol titer (mg patchoulol L⁻¹ culture) in FC-3283 obtained from shaken and static C. reinhardtii cultures after 4 d and 8d. Values are means ±SD (n=5). Asterisks indicate the results of the Student's t-tests: p<0.001 (***) and p<0.0001 (****).

FIG. 7A shows liquid-liquid extraction of FC-3283 (bottom layer) with 96% ethanol (upper layer). FIG. 7B shows representative GC-FID chromatograms of liquid-liquid patchoulol extraction samples, with a clear patchoulol peak (20.4 min) appearing in FC-3283 before extraction and in ethanol after extraction.

FIG. 8A shows chemical structures and properties of commercial FC liquids and dodecane that were investigated in this work. Indicated underneath are GC-FID peak areas of different terpenoids after 10 days of 2-phase living C. reinhardtii extraction cultures with each solvent. Peak areas represent mean±SD (n=5). The chemical structures of FC liquids were obtained from published literature (FC-40:33 FC-770 & FC-328334). All chemical structures were drawn using ChemDraw v20.1. FIG. 8B shows representative GC-FID chromatograms of FC-40, FC-70 and FC-3283 underlays after 10 days of 2-phase solvent-medium culturing with different engineered terpenoid-producing C. reinhardtii strains. FIG. 8C shows extraction efficiency of various terpenoids accumulated in different FCs after accumulation from two-phase engineered algal culture. Liquid-liquid extraction with 96% ethanol (1:1 v/v) for 16 hours was performed on FCs. Grey bars indicate the proportion (%) of terpenoid concentration observed in ethanol after extraction compared to starting concentration in each FC.

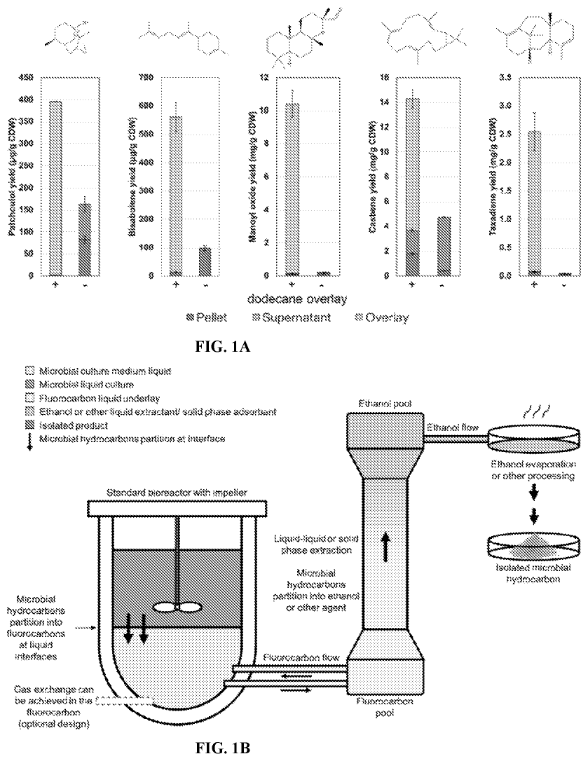

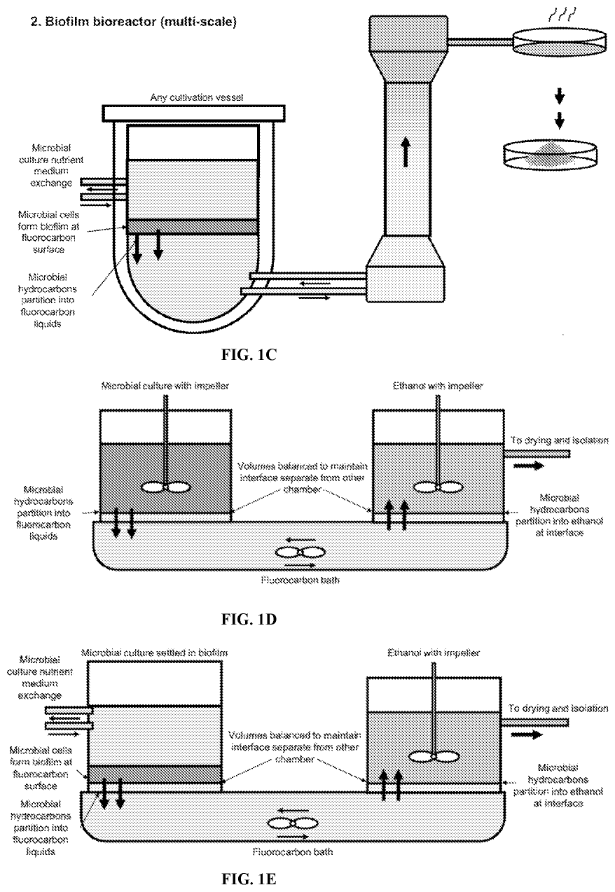

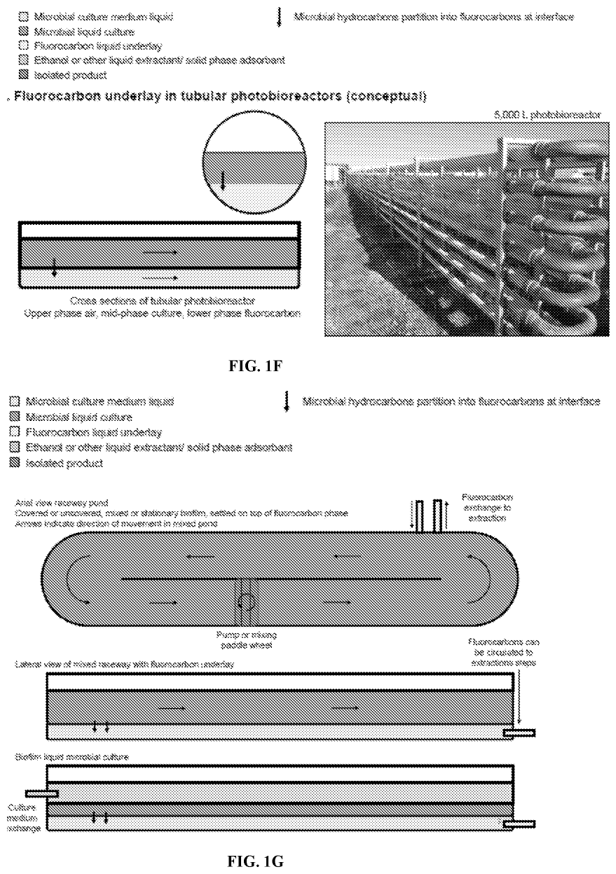

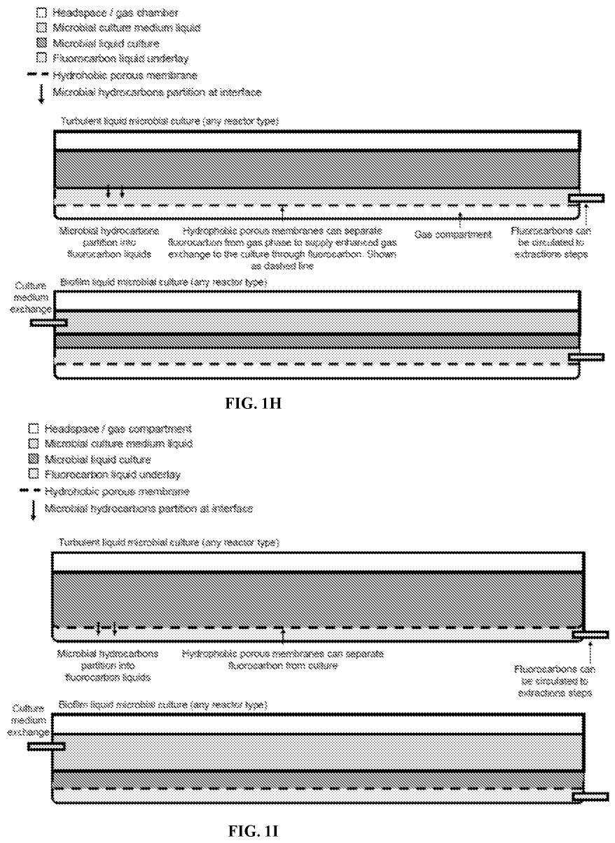

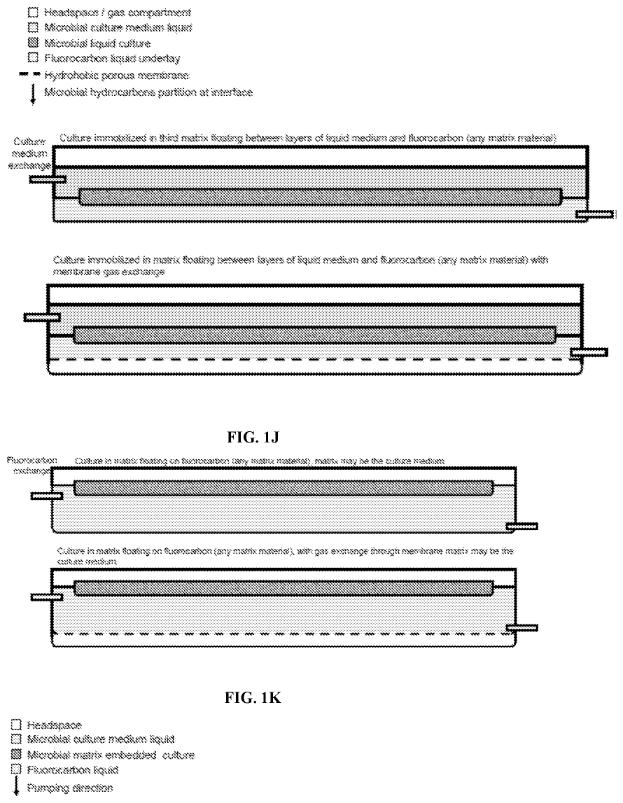

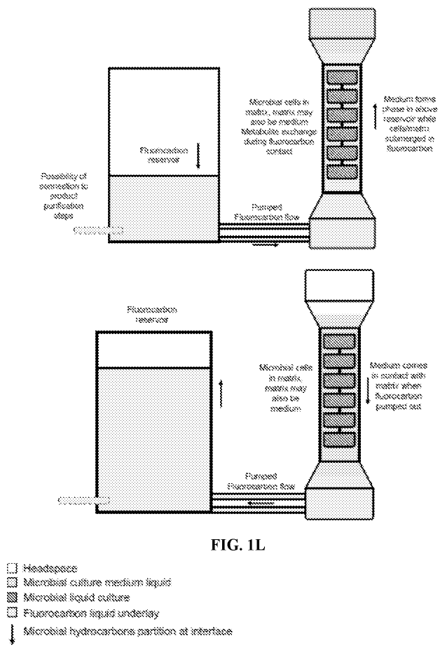

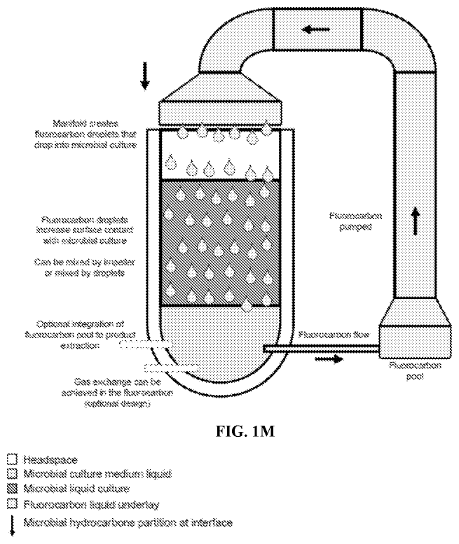

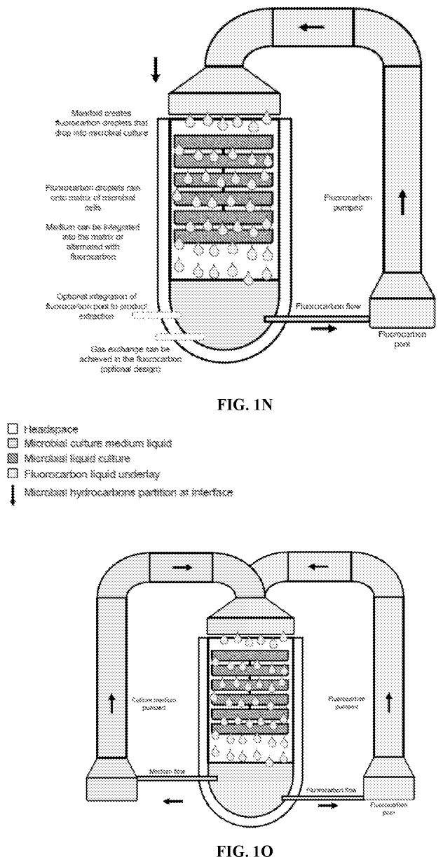

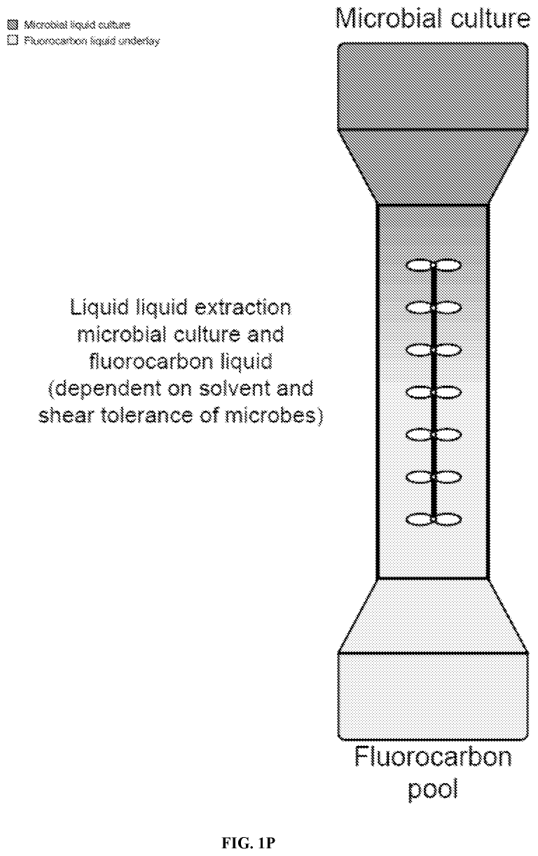

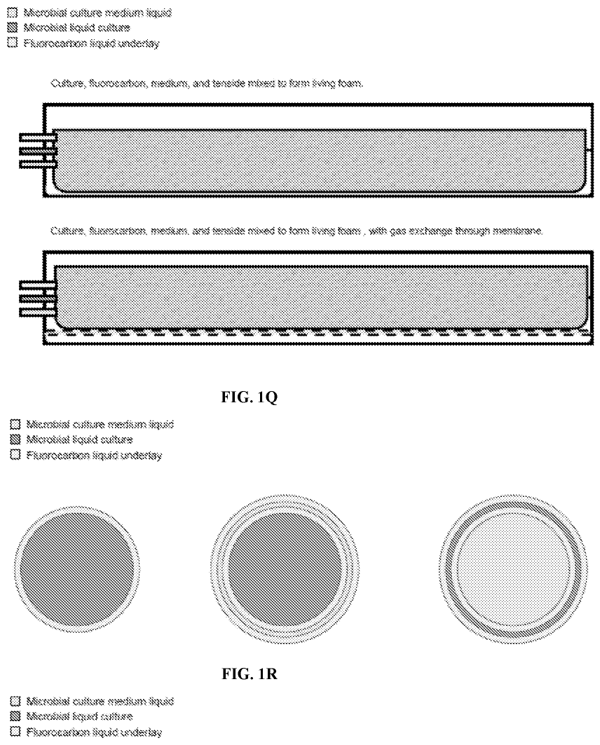

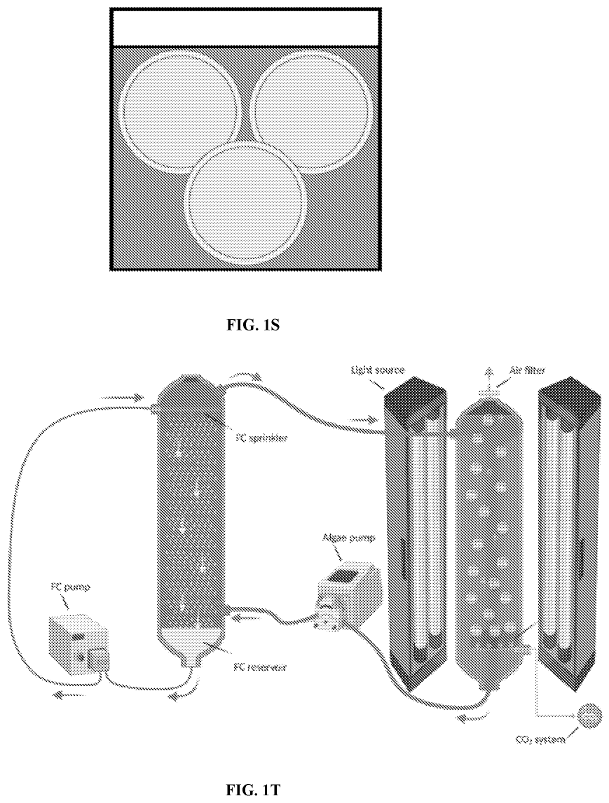

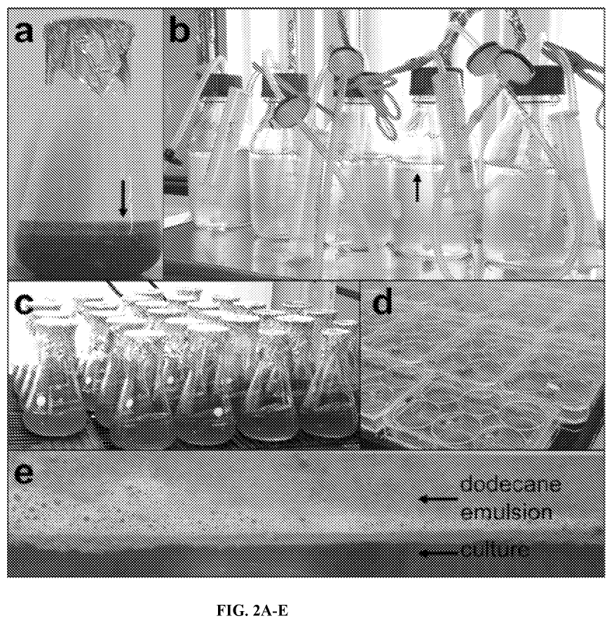

### DETAILED DESCRIPTION OF THE INVENTION

The disclosed method and compositions may be understood more readily by reference to the following detailed description of particular embodiments and the Example included therein and to the Figures and their previous and following description.

The methods and systems disclosed herein are based on the discovery that some longer chain fluorocarbons that are liquid in operational temperature ranges used for microbial culture and which are denser than water, can also be used for live extraction of chemicals (such as terpenes) which accumulate in hydrophobic solvents, from microbial cultures. These certain fluorocarbons have living extraction behaviors as bio-compatible solvents like dodecane but sit underneath the liquid culture instead of on top of the microbial culture, and provide many advantages not seen when extracting with dodecane. In addition, the environmental safety and non-toxicity of these compounds is much greater than the alkane counterparts, which are flammable and although immiscible in water, not environmentally safe.

### II. COMPOSITIONS

The disclosed compositions and methods are applied in microbial cell cultures for extraction of chemicals which are soluble in hydrophobic solvents. Therefore, in one embodiment, the composition containing the metabolite of interest is not a plant material. “Plant material” as used herein not only includes materials which are essentially unprocessed and as such are clearly recognizable as being of plant origin, for example bark, leaves, flowers, roots and seeds, but also materials, which although originating from plants, have been subjected to various processes and as such have a form which is somewhat different than the plants from which they originated, for example ground, dried roots or seeds, such as ground cumin and ground ginger, and expressed oils. Plant material also includes plant tissue culture cells (those treated by hormones to be in the form of undifferentiated callus and cultured in liquid medium like microbes or moss. Moss is also a plant that could be cultured with fluorocarbon underlay.

### A. Microbial Culture Systems

All organisms share similar blueprints in their cellular metabolism, stemming from common evolutionary origins. In the environment, some organisms have evolved specialty chemical production by modifying central metabolites for different reasons, namely defense. Humans have found use for many of these natural products as medicines, fragrances, biofuels, or specialty chemicals¹. Using genetic engineering, it is possible to transfer the metabolic pathways from higher organisms into easier to handle microbes². Microbes are single or filamentous cells that are grown in liquid nutrient solution called medium. With agitation by stirring or shaking, they can double indefinitely so long as nutrients are provided. Microbes have a finite ability to accumulate non-native compounds in their membranes and compartments but can effectively shed those compounds and make more when a physical sink is present to ab/adsorb the compounds from the cells.

In one embodiment, the microbial or cell-culture system produces the metabolite of interest naturally and in other embodiments, the microbe or cell culture is genetically engineered to produce the metabolite. Exemplary microbes in the microbial culture systems include, but are not limited to bacteria, yeasts, filamentous microbes, plant callus culture, moss culture and algae. Strains of the green model microalga Chlamydomonas reinhardtii that had been previously engineered to produce the sesquiterpenoid patchoulol²³ the diterpenoids taxadiene, casbene, or 13R(+) manoyl oxide,^{24,} are known in the art.

Many isoprenoids are naturally produced in small quantities and may be found on slow-growing, non-farmable, or environmentally sensitive organisms.⁸ The biological universality of isoprenoid precursors IPP and DMAPP enables the transfer of modular terpene pathways from progenitors to microbial hosts by the heterologous expression of their terpene synthases and P450s.⁹

Metabolic engineering of microbes for the production of heterologous isoprenoids is now a mature technology, with several commercial enterprises converting base feedstocks into hydrocarbons of various complexities through engineered organisms.¹ Hemi-(C5), mono-(C10), sesqui-(C15), di-(C20), and tri-(C30), non-canonical terpenoids (C11, C12, C16, C17), and carotenoids² are now routinely produced in diverse host organisms such as bacteria, yeast, microalgae, and mosses.³⁻⁶ These engineering successes leverage the universal need for isoprenoid precursors isopentenyl and dimethylallyl diphosphate (IPP and DMAPP), and their subsequent condensed and dephosphorylated hydrocarbon chains. Naval, et al., reviews the detailed description of various approaches used for engineering of methyl-D-erythritol-4-phosphate (MEP) and mevalonate (MVA) pathway for synthesizing isoprene units (C5) and ultimate production of diverse isoprenoids. The review particularly highlighted the efforts taken for the production of C5-C20 isoprenoids by metabolic engineering techniques in E. coli and S. cerevisiae over a decade (Appl. Microbiol. Biotechnol., 2021, 105, 457-475; See also, Deletos, et al., Trends in Biotechnology, July 2020, Vol. 38, No. 7).

Microbial culture can be a liquid culture, a microfilm culture or a culture of microbial cells grown on a solid substrate such as a matrix or a foam, or microdroplet culture systems.

### B. First Extraction Composition

The first extraction composition is preferably a solvent which is denser more-dense than water, allowing for formation of an under layer-or ‘underlay’-in microbial cultures, is biocompatible, non-toxic and non-environmentally hazardous, and are preferably liquid at biologically relevant temperatures. Biologically relevant temperatures as used herein refers to the temperature at which the microbial culture is maintained for microbial cell growth and metabolite production. The first extraction composition is preferably a substituted hydrocarbon.

i. Fluorocarbons

Fluorocarbons are man-made chemicals based on the replacement of hydrogen atoms in hydrocarbons with fluorine atoms. These fluorocarbons have numerous chemical formulas, with increasing carbon chain lengths, linear/circular forms, as well as linkages to other chemical groups. Shorter chain-length fluorocarbons have low boiling points and are gaseous at SATP (standard atmospheric temperature and pressure). By contrast, higher chain-lengths are liquids at SATP which are denser than water and form a separate phase underneath when combined in a container.

Fluorocarbons are variants of hydrocarbons where the H atoms have been replaced by fluorine (F) atoms. These compounds are called fluorocarbons or perfluorocarbons. Although short chain gaseous perfluorocarbons are considered significant atmospheric climate change agents, those with larger molecular masses are liquid at room temperature and atmospheric pressure. They are completely non-reactive, non-flammable, non-toxic and denser than water. In a preferred embodiment, the perfluorocarbon is a liquid at biologically relevant temperatures. Biologically relevant as used herein refers to the temperature used to cultivate the microbe being used to produce the product of interest. For example, the perfluorocarbon can be a liquid at a temperature below 80° C. for example, at room temperature. Because of their density, liquid FCs form a hydrophobic underlay phase with aqueous solutions, like culture medium. Fluorocarbons liquids (FC) come in many forms, some cyclic and some based on different conjugations with atoms such as nitrogen. Below are chemical structures of the traditional solvents hexane (C6) and dodecane (C12) for reference, two types of structure are shown, illustrating carbon atoms with hydrogen bonds. Four commercial FC compounds available from the company 3M are also shown, FC 3283, FC 40, FC 770, and FC 3284. These are used in cooling electronics equipment heat-transfer applications.

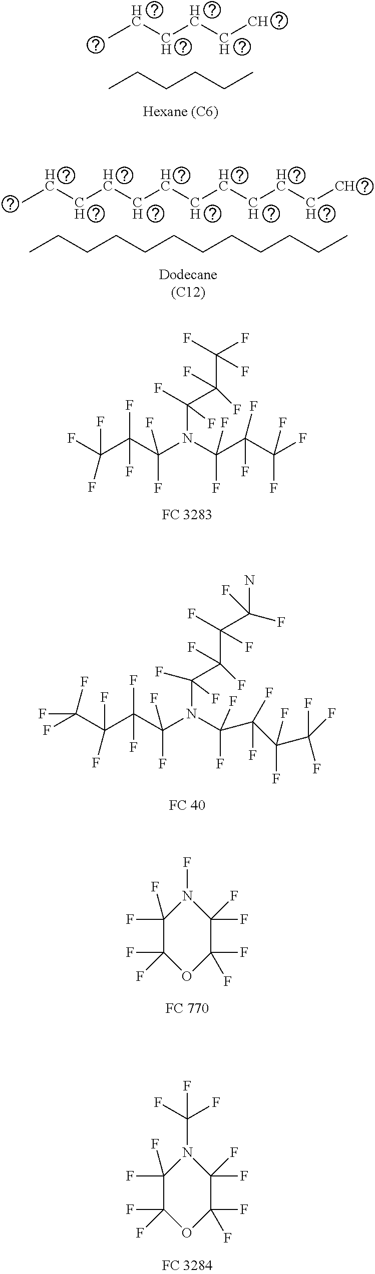

These FC compounds are more dense than water, and the water floats on top of them compared to dodecane which floats above the water. In addition, they are largely inert and biologically non-toxic.

These FC compounds are more-dense than water, and the water floats on top of them compared to dodecane which floats above the water. In addition, they are largely inert and biologically non-toxic.

Studies disclosed herein have determined that liquid solvents such as FCs, for example, FC 3283 and FC 40, can be used also as a physical sink for engineered hydrocarbons from microbial cell culture during growth without destruction of the cells. The FC can be used to “milk” hydrocarbons from growing cultures. The FCs milk hydrocarbons from living microbial cultures in a non-destructive and chemically inert manner while removing the risks and issues of hydrocarbon solvents. They are then amenable to isolation of the extracted hydrocarbons simply with common purification strategies like solid-phase or liquid-liquid solvent exchange. FCs provide a room-temperature, inert, milking solution for microbial cultures, which can overcome the emulsion issues of turbid cultivation strategies by avoiding surface disturbances like bubbling and shaking with the culture interface. As FCs are under the culture, emulsion and physical issues of traditional solvents caused by turbid bubbling can be avoided. Underlay-culture surfaces also allow the settling of cells on the interface surface, which may enable better product transfer with less energy in mixing. An additional benefit to metabolite extraction with liquid FCs is that they are also immiscible with solutions like ethanol and other solvents. This makes them readily adaptable to solid-phase and liquid-liquid downstream purification steps to recover isolated specialty chemicals. Hydrocarbon solvents do not do this. FCs, therefore, provide a room-temperature, inert, milking solution for microbial cultures that allows new bioreactor designs to exploit this underlay principle.

TABLE 1
Comparisons of chemical properties of perfluorocarbons (FCs),
note FC-3284 evaporates at room temperature and is not suitable as an
underlay, except in specialized sealed containers. 1,1,1,2-
tetrafluoroethane is shown for comparison, but is gaseous and not suitable

| | Perfluorocarbon (PFC [symbol] ) | | | | Hydrofluorocarbon (HFC) |
|---|---|---|---|---|---|
| | FC-40 | FC-770 | FC-3283 | FC-3284 | 1,1,1,2-Tetrafluoroethane |
| Appearance | Clear, | Clear, | Clear, | Clear, | Colorless |
| | colorless | colorless | colorless | colorless | Gas *not suitable as underlay |
| | liquid | liquid | liquid | liquid | |
| Chemical formula | [symbol] | [symbol] | [symbol] | [symbol] | [symbol] |
| Avg. molecular | 650 | 399 | 521 | 299 | 102.3 |
| weight, g/mol | | | | | |
| Boiling point, ° C. | 165 | 95 | 128 | 50 | −26.3 |
| Pour point, ° C. | −57 | −127 | −65 | −73 | −103 |
| Liquid density, g/cm² | 1.855 | 1.793 | 1.820 | 1.710 | 0.00125 |
| | | | | | (gas) |
| Ozone | 0 | 0 | 0 | 0 | insignificant |
| depletion | | | | | |
| potential | | | | | |

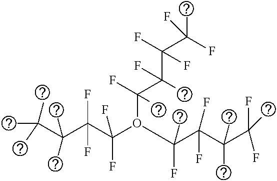

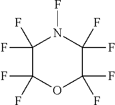 [SMILES: FN1C(F)(F)C(F)(F)OC(F)(F)C1(F)F]

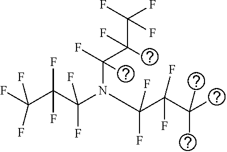 [SMILES: FC(F)(F)C(F)(F)C(F)(F)N(C(F)(F)C(F)(F)C(F)(F)F)C(F)(F)C(F)(F)C(F)(F)F]

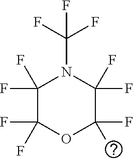 [SMILES: FC(F)(F)N1C(F)(F)C(F)(F)OC(F)(F)C1(F)F]

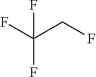

[symbol]

indicates data missing or illegible when filed

In preferred embodiment, the fluorocarbon is not a gas, or R-134a, which is a fluorinated hydrocarbon also known as Freon, which is a gas at room temperature. PURE5 Extraction with R-134a uses pressure and room temperature extraction, which extracts the terpenes and the cannabinoids from plant tissue.

ii. Other Substituted Hydrocarbons

While liquid fluorocarbons are exemplified herein, other substituted hydrocarbons exist with chlorine, bromine, and iodine atoms which may be used like FCs, in the disclosed methods. Tetrachloroethylene (illustrated below) is commonly used as a liquid solvent for dry-cleaning, it is more-dense than water, liquid at room temperature, and could work similarly to FCs in microbial extraction. Similarly, tetrabromoethane (below) is interesting in this regard.

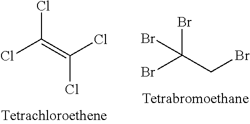 [SMILES: ClC(Cl)=C(Cl)Cl ; BrCC(Br)(Br)Br]

### C. Second Extraction solvent/composition

The methods and systems disclosed herein use a second extraction composition to extract the metabolite from the first extraction composition. Second extraction compositions preferably include other environmentally friendly chemicals such as ethanol, methanol, acetone, etc., as a second extraction solvent, for liquid/liquid extraction. In some embodiments, the second extraction composition is a solid. For example, silica-based solid phase extraction (SPE) can be used to adsorb/isolate patchoulol and similar terpenoids from the solvent due to its hydroxyl group. After binding terpenoids to silica, they can be eluted again with ethanol.

### D. Metabolites

The disclosed methods and systems can be used to recover any metabolite of interest from a microbial culture, so long as the metabolite can accumulate in a hydrophobic solvent. Examples include, but are not limited any hydrophobic metabolite, such as isoprenoids, steroids, phenolics, alkaloids, etc.

Isoprenoids, also called terpenoids, are a large and highly diverse family, of natural products with important medical and industrial properties. Plant terpenoids are used for their aromatic qualities and play a role in traditional herbal remedies. Terpenoids contribute to the scent of eucalyptus, the flavors of cinnamon, cloves, and ginger, the yellow color in sunflowers, and the red color in tomatoes. Well-known terpenoids include citral, menthol, camphor, salvinorin A in the plant Salvia divinorum, the cannabinoids found in cannabis, and ginkgolide and bilobalide found in Ginkgo biloba. The provitamin beta carotene is a terpene derivative called a carotenoid. Terpenoids can be classified according to the number of isoprene units that comprise the parent terpene: hemiterpenoids (examples, DMAPP, isopentenyl pyrophosphate, isoprenol, isovaleramide, isovaleric acid, HMBPP, prenol); monoterpenoids (examples, bomyl acetate, camphor, carvone, citral, citronellal, citronellol, geraniol, eucalyptol, hinokitiol, iridoids, linalool. menthol, thymol); sesquiterpenoids (examples, Famesol, geosmin, humulone, patchoulol); diterpenoids (examples, Abictic acid, ginkgolides, paclitaxel, retinol, salvinorin A, sclareol, steviol, taxadiene, casbene, or 13R(+) manoyl oxide); sesterterpenoids (Andrastin A, manoalide); triterpenoids (examples, amyrin, betulinic acid, limonoids, oleanolic acid, sterols, squalene, ursolic acid); tetraterpenoids (carotenoids); polyterpenoid (example, gutta-percha-natural rubber).

### II. METHODS

This disclosure covers the use of perfluorocarbon liquids as bio-compatible and safe solvents for in-line milking of engineered microbial or cell-cultures that produce specialty isoprenoid hydrocarbon products.

Many microbes produce hydrocarbon products of value, both engineered and natural. The advent of synthetic biology and metabolic engineering is now creating portfolios of microbes which produce a range of specialty chemicals such as medicines, fragrances, and biofuels in fermentative (bacteria, yeasts), or photoautotrophic cultivation concepts (algae, moss, cyanobacteria). Many organisms have been shown to be amenable to metabolic engineering for the production of specialty chemicals, including bacteria, yeasts, and algae. Regardless of microbe type, these cells have limited capacity for accumulation of heterologous products as there are no compartments or tissues for them to accumulate in. Microbes are grown in culture medium, which is aqueous, containing the nutrients required for microbial growth. Traditional metabolite extraction processes such as those described in WO 2008/071985 subject isolated microbial biomass or organisms tissues to solvents like hexane acetonitrile, or methanol, in a process wherein the organism is destroyed in order to extract a desired chemical compound.

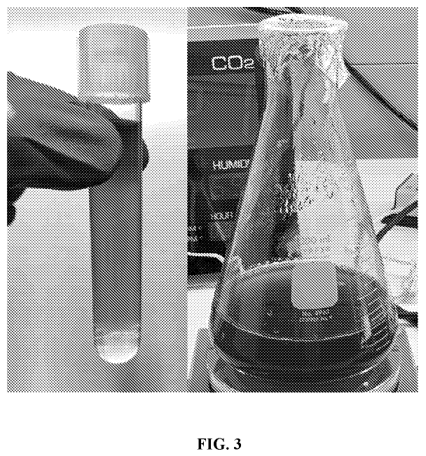

At lab-scale, liquid-liquid interactions of microbial culture with bio-compatible solvent overlays allow direct non-destructive extraction of high-value chemicals from cell membranes in a process called ‘milking’. However, this type of extraction is not scalable due to issues of emulsions at the gas-culture-solvent interface, and the risks/process complications of hydrocarbon solvents. In lab scale, this kind of non-natural product ‘milking’ is achieved using an organic (hydrocarbon), bio-compatible solvent layer such as decane³ (C10), dodecane⁴ (C12), hexadecane (C16), or heptadecane (C17). These solvents float on top of a microbial culture (FIG. 3, black arrow a/b) and are related to the common hexane (C6) which is used for cell disruption and extractions in analytical settings but differ in that they are non-toxic to most cells unless heavily agitated in them and act as a physical sink for metabolite accumulation. For example, different specialty chemicals are able to partition into dodecane at different efficiencies based on their hydrophobicity, but for all products tested, higher yields are achieved when dodecane is added to culture as a physical sink.

Hydrophobic overlays pose the technical challenge that they are not scalable, as turbid microbial cultures with bubbling or intense mixing result in hydrophobic cell products, i.e. cell walls/proteins, mixing with solvents to form an emulsion (FIG. 3E) limiting extraction potential and downstream processing. There are also dangers associated with large volumes of flammable solvents at scale, in addition to their incompatibility with most plastics/tubing. Intensive mixing of cells is also required against gravity sedimentation to keep cells in contact with the solvent interface.

The disclosed methods are based on the discovery that solvents which are more-denser than water, allowing for formation of an under-layer or ‘underlay’ in microbial cultures, are biocompatible, non-toxic and non-environmentally hazardous, and are liquid at biologically relevant temperatures used to culture microbes, exemplified herein using biologically inert perfluorocarbon liquids, can be used in microbial milking, and most interestingly, form a hydrophobic layer under cultures due to their densities.

As the extraction solvent is under the liquid culture, new possibilities of advanced cultivation concepts for microbial milking are conceivable. The inert nature of the solvents such as perfluorocarbons are far safer than other solvents and generate high-purity, native-state metabolites without complexities in downstream processes.

The disclosed systems and methods are organism agnostic and could be applied to bacteria, yeasts, filamentous microbes, moss and plant-cell culture, and algae.

The disclosed methods allow for continuous natural milking of microbial culture without tissue disruption and can produce isolated metabolites in their native states. The products can be readily recovered in a solvent such as ethanol, which, when dried, leaves a natural powder of the target hydrophobic products. The method preferably does not use PURE5 R134a terpene wash, other fluorinated gasses (at SATP) or pressure gradients.

The process includes the steps of (a) contacting the raw material (present in a microbial culture/produced by a microbial culture)/or a microbial culture with a first extraction composition so as to extract the compound or composition of interest from the raw material/microbial culture into the solvent and (b) separating the solvent containing the extracted compound or composition from the raw material. The method also includes a secondary direct extraction step (1 Liquid-liquid/liquid-solid extraction) (c) using other environmentally a second extraction composition, for example other environmentally friendly chemicals such as ethanol as a second extraction solvent, for liquid/liquid extraction, for example. The contacting of the extraction solvent with the raw material to be processed may be carried out under vigorous mixing conditions so as to facilitate the dissolution of the material to be extracted into the extraction solvent. Vigorous mixing may be achieved by mechanically shaking the extraction vessel containing the raw material/extraction solvent mixture, by stirring that mixture, by passing the liquid solvent through a stationary packed bed of solid biomass or by the application of ultrasonic excitation.

The microbial culture is not a homogeneous liquid, rather it is a culture medium (liquid) in which particulate microbes grow and proliferate. Through interaction with the fluorocarbon underlay, hydrophobic metabolites produced by the microbes partition into this phase, driven by physical properties of each metabolite. There is a balance in all bio-reactor/cultivation designs, between the microbial growth and the extraction of metabolite(s). In some cases, metabolites may only be produced during certain growth stages which can be maintained by chemo-/turbidostat, in others, static cultures which leverage the lower phase interface as a settling point for bio-film formation driven by gravity may be possible. This technology is agnostic of the growth condition, meaning that the bio-reactor process and growth design can be tailored to each organism and milking of the product with fluorocarbon achieved.

Operating conditions could range anywhere under the boiling dense solvent depending on the microbe to be cultivated. Microbes have cultivation optima from 4 C-95° C. The disclosed methods and systems expand metabolite extraction to extremophiles which grow at higher temperatures (˜80-90° C.), in which metabolite extraction would not work with traditional solvent overlays. FC 40 and FC 3283 are particularly valuable in this regard, since their boiling points are well over 100° C. There are hundreds of examples of various types of organisms that grow at various temperatures. But the FCs are broadly applicable to them all, by selecting an FC that is liquid at the organisms growth temperature.

Flow rates are determined by the culture volume, the growth rate of the organism, and the growth phase when product is made. For example, a microbe that makes product only when it is doubling, has to be kept healthy and doubling, so medium turnover and purging would be matched to its doubling rate. For organisms that produce products when the cell reaches a resting state, a reactor can be used to grow as much biomass as possible, then interact with the first extraction composition as long as possible while the cells are resting on it. Flow rate of the first extraction composition-second extraction composition for extraction will depend on the concentration of product yielded from the cells. The extraction is preferably at room temperature or around culture operating temperature, depending on the organism used. For higher temperature conditions solid state adsorptions are preferably used.

The disclosed methods not only apply to axenic microbes, but microbial consortia potentially composed of single cells, groups of cells, or filamentous tissues (hyphal/strands, etc.).

After the primary solvent containing the extracted metabolite has been separated from the raw material, the solvent/metabolite is subjected to liquid/liquid or liquid solid extraction using a secondary solvent, following which the metabolite can be recovered from the secondary solvent: the secondary solvent can be removed, e.g. by distillation or flash evaporation, to leave the extract which can be used as it is or sent for further processing, e.g. purification.

In one preferred embodiment, the disclosed method does not use short chain R-134a aka 1,1,1,2-tetrafluoroethane (or other pressurized gasses) which has (ve) been used to extract chemicals of interest from plant tissue as these would constitute destructive processes, meaning, the plant tissue or organism is destroyed/dead/damaged after extraction.

### III. SYSTEMS

Lab-scale cultivation units to test various conformations of underlay and microbial cultures are disclosed.

Milking of metabolites from living culture has been shown at lab scale with bio-compatible high-molecular weight solvents (i.e. dodecane) that are lighter than water and form an overlay over top of the microbial culture, creating a 2-phase living extraction system. Many issues have been observed in the use of lighter solvents at scale, especially their contamination with other chemicals, requirements of specialized infrastructure for their handling, and emulsion formation due to the turbidity and presence of hydrophobic proteins at the culture-solvent interface. These solvents are especially harsh on plastics/silicone and limit their use in non-specialized pumps/tubing/containers.

In comparison to traditional solvents, fluorinated carbon solvents (fluorocarbons) are denser than water and create a 2-phase system with an aqueous upper phase (water/medium) and lower phase (fluorocarbon). The studies herein have determined that liquid fluorocarbons in a 2-phase system underneath a microbial culture are non-toxic to microbial cells and accumulate hydrophobic chemical products generated by the microbial culture. This is a novel approach to traditional solvent overlay milking which could be applied to many organisms: bacteria, archaea, yeasts, filamentous organisms, moss, plant cell culture, algae, and cyanobacteria. The fluorocarbon underlay allows continuous metabolite extraction at room temperature and atmospheric pressure without harming the cells.

The density of liquid fluorocarbons and other solvents/compounds means fluorocarbons also readily phase separate from other solvents. This property enables simple recovery of metabolites in a native state from fluorocarbon phases into other products, like ethanol, and rapid regeneration of the fluorocarbon. Implementation of fluorocarbons to microbial metabolite extraction allows new conceptualization of microbial milking strategies.

Microbes are grown with a fluorocarbon phase underneath which accumulates their native and heterologous hydrocarbon products, followed by in-line extraction of those products from the fluorocarbon. The use of an underlay solvent is a new concept for microbial bioreactors and various proposals for how it can work are given in this document.

In all conditions, the metabolite extraction process is non-lethal to the cells, whether growing or resting state. The first extraction composition can also be used as a reservoir through which gas exchange is conducted, as the gas exchange capacity of these fluids is high. In other iterations, the culture and first extraction composition could be separated by hollow-fiber membranes or porous membranes.

The systems include (a) microbial cell culture vessels such as a fermentation bioreactor or a biofilm bioreactor, (b) one or more extraction vessels and (c) a collection vessel. The microbial cell culture vessel includes a microbial cell population producing the product of interest, and the first extraction solvent as an underlay to the microbial cell population. A portion of the vessel containing the first extraction solvent is connected to the extraction vessel by pipes allowing the flow of extraction solvent to and from the microbial culture vessel and the extraction vessel. At least one of the extraction vessels include the second extraction solvents in one extraction vessel or in separate extraction vessels. The second extraction solvent is in fluid communication with a third collection vessel; thus, the extraction vessel can include an open connection to the collection vessel, allowing for fluid flow from the extraction vessel to the collection vessel.

Specific aspects are shown in FIG. 1B-1I. FIG. 1B-1C show illustrations of a lab scale system for microbial culture with fluorocarbon underlay extraction of microbial produced hydrocarbons/specialty chemicals in a fermentation bioreactor (FIG. 1B) or a biofilm reactor (FIG. 1C). FIG. 1D-1E is a schematic showing lab scale microbial culture with fluorocarbon underlay extraction of lab scale system for microbial culture with fluorocarbon underlay extraction of microbial produced hydrocarbons/specialty chemicals using a mixed floating bath liquid extraction (FIG. 1D) or a stationary floating bath liquid extraction (FIG. 1E). FIG. 1F is a schematic showing for scaled fluorocarbon use in large-scale photo-microbial cultures (algae, cyanobacteria, moss). FIG. 1G is a schematic showing fluorocarbon underlay in raceway ponds. FIG. 1H-1K show a schematic of scaled fluorocarbon use in microbial cultures, with Fluorocarbon underlay on hydrophobic membrane gas exchanger (FIG. 1H), fluorocarbon underlay with culture separated by membranes (FIG. 1I), Fluorocarbon-matrix-medium liquid sandwich (FIG. 1J) or a Fluorocarbon-floating matrix (FIG. 1K). FIG. 1L shows lab scale microbial culture with fluorocarbon underlay using a packed bed matrix with phase exchange (multi-scale) (FIG. 1L), liquid-liquid drop extraction (multi-scale) (FIG. 1M) or a packed bed matrix droplet extractor (multi-scale) (FIG. 1N and FIG. 1O). FIG. 1P shows a standard liquid-liquid extraction directly with fluorocarbons and microbial culture. FIG. 1Q shows fluorocarbon-culture-medium foam. FIG. 1R and FIG. 1S show Fluorocarbon-culture-microdroplet encapsulation (FIG. 1R) and fluorocarbon-medium-microdroplet encapsulation (FIG. 1S). FIG. 1T is a schematic lab scale microbial culture with fluorocarbon underlay by liquid-liquid drop extraction with integrated photobioreactor system (multi-scale).

Generally, the various designs and systems disclosed herein are operated using bioreactor operation and reaction conditions and considerations known in the art to permit microbial growth or the growth of dedifferentiated cell cultures (reviewed in Zhong, Comprehensive Biotechnol. 2: 165-177 (2011)). Details of bioreactor operation for growth many microbes and cell culture types can be found in (Posten 2018 Integrated Bioprocess Engineering, De Gruyter, Inc.,). Microorganisms are often classified according to their growth temperature as either thermophiles (growth temperature: >50° C.), mesophiles (growth temperature: from 20 to 50° C.) , or psychrophiles (growth temperature: <20° C.). Bioreactors are devices that allow control of an environment in which to grow microorganisms or cell culture and may also provide a read-out of growth through various measurements (optical density, pH, metabolite sensors, gas sensors). Bioreactors are operated at the optimal temperature and pH for the microorganisms selected. Microorganisms and cell cultures are grown in liquid chemical solutions composed of either complex ingredients (yeast extract) or pure chemicals, these solutions are called medium. Medium contains nutrients (micro and macro) which enable culture growth as well as a carbon source, which can be organic (i.e. glucose, ethanol, cellulose) or inorganic (i.e. CO2 for photosynthetic organisms, CO, or other gasses depending on the organism). Operation of microbial milking is conducted in cultivation vessels which enable microbial or de-differentiated cell culture growth, which can be any container that holds liquid and provides known in the art conditions for each microbe as outlines in the various figures presented. Solvents that are more-dense than water (here described as perfluorocarbon liquids) are introduced to these vessels as a liquid phase by pouring, decanting, or pumping into the same vessel as the culture medium and cells. Cells are then permitted to grow by procedures known in the art to permit cellular growth (microbes, filaments, or dedifferentiated cell culture), the culture medium is allowed to interact with the dense solvent through any means of physical interaction, some non-exhaustive examples are illustrated in the Figures. As the culture growth proceeds, or the biomass is left in contact with the solvent, extraction and partitioning of the target products into the solvent occurs without harming the cells. The solvent is then subsequently taken and interacted with an extraction method (liquid/liquid or liquid//solid) as described herein.

The next sections describe exemplary reactor designs.

### 1. Applications of Fluorocarbons to Traditional Fermenters

The systems include applications of fluorocarbons to traditional fermenters

In this embodiment, a first extraction solvent such as a fluorocarbon can be added to traditional microbial fermenters as an underlay. (FIG. 1B). Microbial hydrocarbons partition into fluorocarbons at liquid interfaces. Gas exchange can be optionally achieved in the first extraction solvent portion of the bioreactor. The impeller can be positioned appropriately to minimize turbidity at the interface. Using enough fluorocarbon as to create a separate lower phase, this liquid can be independently circulated into a separate extraction vessel, either solid-or liquid-liquid where partitioning coefficient of the product can drive it into a desired phase and facilitate its further extraction/concentration. Shown in FIG. 1B is a concept for liquid-liquid extraction with ethanol. Fluorocarbon can be recycled into the bioreactor.

### 2. Biofilm Bioreactor

Without agitation, microbial culture can form a biofilm at the interface between culture medium and fluorocarbon (FIG. 1C). This is a unique possibility with the first solvent underlay which does not work with other biocompatible solvent overlays. Direct growth in a biofilm may enable better product partitioning into the fluorocarbon. As shown in FIG. 1C, Microbial cells form biofilm at the fluorocarbon surface, and microbial hydrocarbons partition into fluorocarbon liquids. The fluorocarbon can be fed to an extraction strategy from the lower phase.

### 3. Floating Bath Bioreactors

In this design, the microbial culture and the second extraction medium are separated by the fluorocarbon in a container with two chambers over a lower compartment which houses the fluorocarbon (FIG. 1D). The design relies on the filling of the system to levels which balance the buoyancies of culture and second extraction solvent/solid i.e., extractant (ethanol) on the fluorocarbon, and gentle agitation to maintain phase separation.

### 4. Floating Bath With Microbial Biofilm

This is similar to the design of the floating bath reactors, the difference being that microbial culture is allowed to form a floating biofilm on the first extraction solvent surface and microbial culture nutrient medium is exchanged to promote biofilm growth (FIG. 1E).

### 5. Application of Fluorocarbons to Traditional Algal Cultivation: Tubular Photobioreactors

As tubular photobioreactors have long tube channels which expose algae to sunlight, fluorocarbon can be included in these concepts as a separate flowing phase. The holding chamber for gas exchange will in some embodiments be the main accumulation point for fluorocarbons. This design is shown in FIG. 1F.

### 6. Application of Fluorocarbons to Traditional Algal Cultivation: Raceway and Covered Raceway Ponds

Raceway ponds which are the most common and lowest energy form of microbial cultivation are designed to include first extraction solvent (e.g. fluorocarbon) baths underneath cultures (FIG. 1G). These are preferably conducted in covered ponds which minimize evaporation and loss of both culture and first extraction solvent. Cultures in this embodiment can be grown in agitated or biofilm style. An aerial view of a raceway pond is shown in FIG. 1G. It can be covered or uncovered, mixed or stationary. In embodiments including mixing, the system includes a pump or mixing wheel in the microbial culture vessel.

### 7. Hydrophobic Membranes for Supplying Gas Exchange

Since extraction solvents such as fluorocarbons enable ease of gas permeability, the cultivation of microbes on such extraction solvents with a porous hydrophobic membrane support, can permit improved gas exchange in microbial cultivation. This has been shown for algal cultures to permit improved CO₂ loading capacity in dense cultures. This embodiment is shown in FIG. 1H, which can be used with any reactor type. Here, the microbial culture vessel includes a microbial cell layer adjacent to the first extraction solvent layer, which is separated from a gas layer by a hydrophobic membrane. Microbial hydrocarbons partition into fluorocarbon liquids. Hydrophobic porous membranes can separate fluorocarbon from gas phase to supply enhanced gas exchange to the culture through the first extraction solvent. The first extraction solvent containing extracted product is circulated (with pumps that do not contaminate the product/solvent. Examples include peristaltic pumps (at small scale) or reciprocating (i.e., piston-driven) and rotary pumps) to an extraction vessel for extraction of the product.

### 8. Separation of Fluorocarbons and Microbial Culture Through Porous Membranes

This design incorporates the use of porous membranes for phase support, such as hollow-fiber or flat membranes without the use of a gas layer as disclosed for the design incorporating hydrophobic membranes for supplying gas exchange (FIG. 1I). This design can enable higher surface area-volume contact rations between microbial cultures and first extraction solvent, for example in designs incorporating vertical bio-reactors. As shown in FIG. 1H/I: Top: Turbulent microbial culture (stirred or recirculated otherwise) comes into contact with extraction solvent at the hydrophobic porous membrane where the product gets transferred from the culture to the solvent. Bottom: Settled microbial culture forms biofilm on top of the hydrophobic porous membrane where the culture comes into contact with extraction solvent, thereby facilitating the transfer of microbial products from the culture to the solvent.

As shown in FIG. 1J, the culture vessel can in some embodiments include culture immobilized in third matrix floating between layers of liquid medium and fluorocarbon (any matrix material). Examples include any high surface-area spongy materials that can facilitate the growth of microbial cultures. At large scale, industrial ceramic-based packing material for bioreactors could be used. In in other aspects, the culture vessel can include culture immobilized in matrix floating between layers of liquid medium and fluorocarbon (any matrix material) with membrane gas exchange (exchange of CO₂ and O₂). FIG. 1J shows top panel shows in one embodiment, the culture vessel containing culture in matrix floating on fluorocarbon (any matrix material), matrix may be the culture medium, and in another embodiment (bottom panel) the culture vessel containing culture in matrix floating on fluorocarbon (any matrix material), with gas exchange through membrane matrix may be the culture medium. As shown in FIG. 1J, the microbes get their nutrient from a liquid culture medium, whereas in 1K, the microbes are immobilized in a solid culture medium (e.g., agar). FIG. 1L shows an embodiment in which the microbial culture vessel includes a packed bed matrix with phase exchange (multi-scale) and it is commented to reservoir containing the first extraction solvent. FIG. 1L shows a (tidal) reactor design where the FC underlay gets regularly pumped in an out of the reactor that houses the microbial matrix. Top panel: As the FC replaces the culture medium and comes into contact with the microbes, the FC extracts product. Bottom panel: As the FC gets pumped out again, the microbes are instead submerged in medium and can acquire their nutrients from it.

In some aspects, the disclosed system includes a manifold in contact with the microbial culture vessel, through which the first extraction solvent is pumped into the microbial culture vessel, resulting in droplets of first extraction solvent contacting the microbial culture (FIG. 1M). Fluorocarbon droplets increase surface contact with microbial culture, and they can be mixed by an impeller. There are two options to purify the product from the FC:. 1). If there is no additional feature integrated, the FC would be recovered by physical means at the end of the extraction process.

Subsequently, the product could be purified using liquid-liquid extraction with ethanol or similar solvents. 2) If there is another feature integrated in the system, a pipe could lead from the FC reservoir to a separate container of e.g. ethanol where the FC comes into contact with the ethanol, the product gets transferred to the ethanol and the “clean” FC gets transferred back to the main reactor where it comes into contact with the culture again. Of note, the FCs are recyclable in this process, after the target molecule is separated, the FC is re-used. The aspect shown in FIG. 1N incorporates a matrix of microbial cells generating the desired product. First extraction solvent (fluorocarbon) droplets rain onto matrix of microbial cells. The embodiment in FIG. 1O shows packed bed matrix droplet extractor (multi-scale), incorporating a cell culture medium pump and a first extraction solvent pump. In some iterations, microbial culture will be embedded into a matrix, such as a sponge material and suspended by supports in an air chamber. In such a situation, culture medium is dropped through the sponge support to provide nutrition to the microbial cultures. In such a reactor, fluorocarbon could be simultaneously dropped with medium to milk produced molecules in a mixture from the cultures while they receive liquid medium. In a lower receiving vessel, phase separation between fluorocarbon and medium can occur to allow independent pumping, or extraction of the fluorocarbon. FIG. 1Q shows an embodiment in which the microbial culture vessel includes microbial cells, first extraction solvent, microbial cell culture medium, and tenside mixed to form living foam (top panel) or incorporating gas exchange through a membrane (bottom panel). FIGS. 1R and 1S show the encapsulation of droplets in different configurations. FIG. 1R left: Individual liquid culture droplets are encapsulated by an FC layer. FIG. 1R

Middle: Individual liquid culture droplets are encapsulated by an FC layer, followed by a culture medium layer and an FC layer again. FIG. 1R Right: Individual culture medium droplets are encapsulated by an FC layer, followed by a liquid culture layer and an FC layer again. FIG. 1S: Individual culture medium droplets encapsulated by FC are kept in a liquid culture pool. FIG. 1T shows a bioreactor consisting of two connected parts.

Right side: Liquid culture flows into the column at the top, gets sparged with CO2 and exposed to light (to facilitate photosynthesis), and exits the column at the bottom.
Left side: The liquid culture enters the column at the bottom. As the culture travels upwards, it comes into contact with FC droplets which causes terpenoid product to get transferred from the culture to the FC.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### EXAMPLES

### Materials and Methods

### Chemicals

Three perfluoro liquids, hereinafter referred to as FCs, were used in this work: the perfluorinated amine Fluorinert™ FC-40 was purchased from Sigma-Aldrich (Overijse, Belgium), while the perfluorinated ether FC-770 and the perfluorinated amine FC-3283 were obtained from Acros Organics (Geel, Belgium). Ethanol (96% vol.) and n-dodecane ($99%) were purchased from VWR International (Fontenay-sous-Bois, France). Prior to its use, dodecane was passed through a Supelclean™ LC-Si solid-phase extraction (SPE) column (Product no. 505374; Sigma-Aldrich, Taufkirchen, Germany) to remove impurities.

### Microalgal Cultures and Growth Conditions

Four strains of the green model microalga Chlamydomonas reinhardtii that had been previously engineered to produce the sesquiterpenoid patchoulol,²³ the diterpenoids taxadiene, casbene, or 13R(+) manoyl oxide,²⁴ as well as their parental strain (‘UVM4’ hereafter termed wild type),2⁵ were used in this work to investigate the terpenoid-extraction potential of perfluoro liquids from two-phase solvent-medium living microbial cultures. All algal cultures were maintained routinely on Tris acetate phosphate (TAP) 26 agar plates under ˜50 μmol m⁻² s⁻¹ light intensity before transfer into 24-well plates containing 1 mL TAP medium, and shaken at 190 rpm on a 12 h: 12 h dark: light (˜150 mmol m⁻² s⁻) cycle. After 2 d, an additional 500 mL of medium was added to each well to replenish nutrients. At 4 d, 400 mL of each culture was inoculated in 40 mL TAP in Erlenmeyer flasks at 120 rpm under the light conditions indicated above and used for subsequent experiments.

### Culture Growth Measurements

Culture growth was measured by recording cell densities using flow-cytometry with an Invitrogen Attune NxT flow cytometer (Thermo Fisher Scientific, UK) equipped with a Cytkick microtiter plate autosampler unit. Prior to analysis, each biological triplicate sample was diluted 1:100 with 0.9% NaCl solution. Of each diluted sample, 250 mL was measured in technical duplicates (n=2) using a 96-well microtiter plate loaded into the autosampler. Samples were mixed three times each immediately before analysis, and the first 25 mL of sample was discarded to ensure stable cell flow rate during measurement. Data acquisition stopped when 50 mL from each well was analyzed. All post-acquisition analyses and population clustering were performed using Attune NxT Software v3.2.1 (Life Technologies, USA). Mean cell concentrations of shaken and static cultures were compared against each other by performing Student's t-tests (one test per sampling point) using the software JMP Pro 16.2 (SAS Institute Inc., Cary, NC).

Algal culture health in contact with dodecane or perfluoro liquids was measured by the variable chlorophyll fluorescence of photosystem II (PSII) with a Pulse Amplitude Modulation (PAM) fluorometer (Mini-PAM-II: Heinz Walz GmbH, Germany). Before each measurement, microtiter plates containing cultures were dark-adapted at room temperature for 15 min to ensure all PSII reaction centers were open. For each sample, the signal amplitude was adjusted before one single-turnover measurement was recorded per sample. The maximum photochemical efficiency (Fv/Fm) was determined using the following relationship (eqn 1), where Fm and FO are the maximal and minimal PSII fluorescence of dark-acclimated C. reinhardtii cells, respectively:^{27,28}

$$\frac{{F}_{V}}{{F}_{m}} = \frac{{F}_{m} - {F}_{0}}{{F}_{m}} \qquad ( 1 )$$

### Terpenoid Capture From Microalgal Cultures

Two-phase living extractions were performed with dodecane, FC-40, FC-3283, and FC-770 by gently pipetting 500 mL of each compound onto 4.5 mL liquid cultures of C. reinhardtii immediately after inoculation. Dodecane instantly formed an upper overlay' phase as reported in several previous studies, 18,23 while FC compounds sank under cultures to form ‘underlays’. FC compounds, however, resembled large bubbles under growing cultures and did not spread as evenly as dodecane on the surface, likely due to the liquid dynamics of water pressing on top of the FC liquids. The two-phase cultures were grown for 10 d in triplicates in 6-well microtiter plates on laboratory shakers as described above.

### Liquid-Liquid Extractions

To test whether the extracted patchoulol could be readily isolated from FC-3283 and enable fluorocarbon recycling, liquid-liquid extractions with ethanol was performed. Specifically, after the terpenoid capture experiments, the FC-3283 underlay from each microtiter well (˜500 mL) was recovered and transferred into a separate 1.5 mL ° FC-3283 from algal cell debris. From each tube, 250 mL of the clean FC-3283 fraction was pipetted into a clean 2 mL microcentrifuge tube to which 250 mL of ethanol (96% vol.) was added. The fluorocarbon/ethanol mix was shaken at room temperature for 16 h at 1000 rpm using an Eppendorf Thermomixer C (Eppendorf AG, Germany) to perform the liquid-liquid extraction. After the extraction, each sample was centrifuged at 3500×g for 5 min to separate the two liquid phases. From each sample, 90 mL of both fractions were pipetted into separate gas chromatography (GC) vials and stored at −20° C. until further analysis as described below.

### Gas Chromatography

Dodecane and perfluoro liquids fractions were analyzed using a gas chromatograph equipped with a mass spectrometer and a flame ionization detector (GC-MS-FID) with minor modifications to a previously described protocol.²³ Briefly, the GC-MS-FID analyses were performed using an Agilent 7890A gas chromatograph connected to a 5975C inert MSD with triple-axis detector. The system was equipped with a DB-5 MS column (30 m×0.25 mm i.d., 0.25 mm um thickness) (Agilent J&W, USA). The temperature profile was set to: injector (250° C.), interface (250° C.), and ion source (220° C.). 1 mL of sample was injected in splitless mode with an autosampler (Model G4513A, Agilent). Column flow was kept constant at 1 mL min⁻¹ with helium as a carrier gas. The initial GC oven temperature of 80° C. was held for 1 min, then raised to 120° C. at a rate of 10° C. min⁻¹, followed by 3° C. min⁻¹ to 160° C., and further to 240° C. at 10° C. min⁻¹, which was held for 3 min. Mass spectra were recorded after a 13.2 min solvent delay using a scanning range of 50-750 m/z at 20 scans per s.

Gas chromatograms were evaluated with MassHunter Workstation software version B.08.00 (Agilent Technologies, USA). The NIST library (National Institute of Standards and Technology, Gaithersburg, MD, USA) was used to identify patchoulol, along with verification using a purified patchoulol standard (Item: 18450, Cayman Chemical Company, MI, USA). Standard calibration curves in the range of 1-200 mM patchoulol in dodecane and FC-3283 were used for quantification. In dodecane samples only, 250 mM of a-humulene was additionally applied as internal standard. Extracted-ion chromatograms (XIC) with mass ranges of 91.00, 138.50, and 223.00 were used for samples with internal standard, and 138.00 and 222.00 for samples without internal standards. All GC-MS-FID measurements were performed in duplicate, and chromatograms were manually reviewed for quality control.

### Results and Discussion

Microbial engineering requires design-build-test-learn cycles as an iterative process of genetic engineering steps to identify optimal combinations of elements to drive cellular flux towards a desired product. These iterations require lengthy trans-formation and phenotypic screening that can be resource-heavy and laborious. Our engineering efforts in C. reinhardtii have relied on the use of dodecane for solvent-culture two-phase extraction of these products, which has served as a bio-compatible compound that can be readily separated and used directly in chromatography.³Recently, it was shown that individual cells or groups of cells can be handled through encapsulation in microfluidic droplets to enable phenotyping without the need for selection agents.²⁹ It was surprising to note that the perfluoro liquids used to make these droplets form a dense under-layer to the culture medium in large quantities. When incubated with our engineered C. reinhardtii cultures, the accumulation of heterologous isoprenoids in these perfluorocarbons was observed. Here, the studies report on and characterize the suitability of perfluoro liquids (FCs) as alternatives to currently used petroleum solvents for microbial cell isoprenoid milking.

### Potential of FC-3283 to Extract Terpenoids From Growing Microbial Cultures

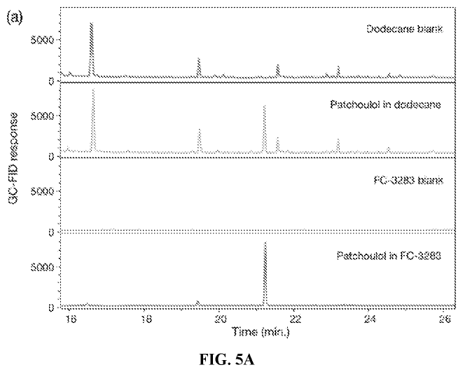

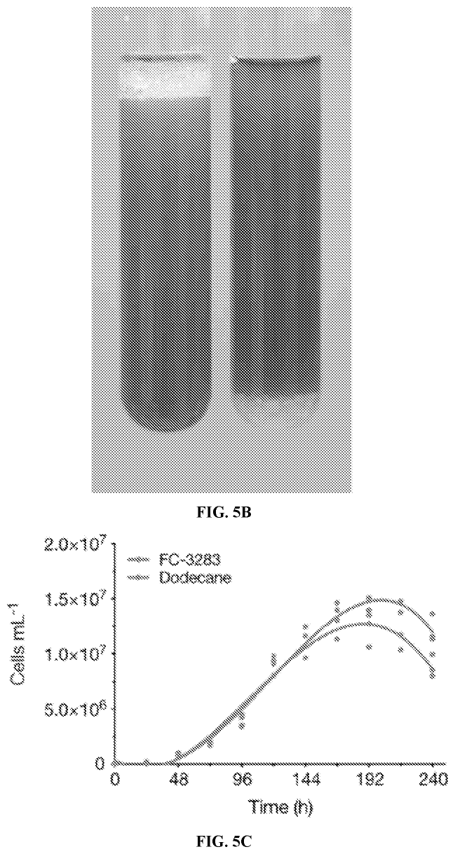

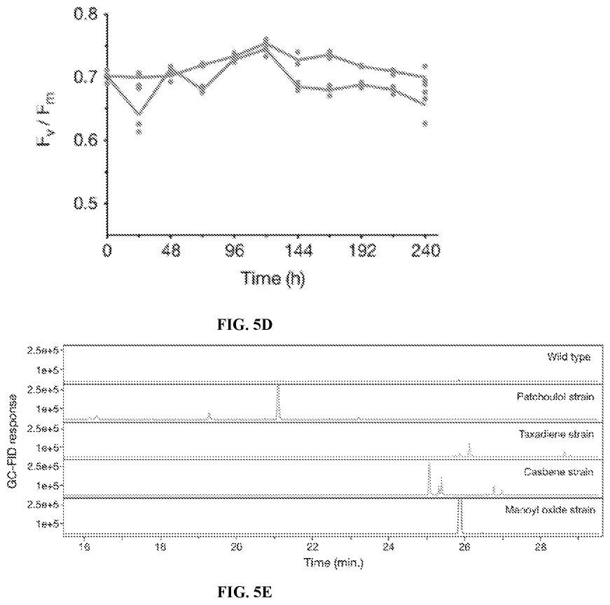

A C. reinhardtii strain previously engineered to produce the heterologous sesquiterpenoid alcohol patchoulol²³ was cultivated with either a dodecane overlay or a commercially avail-able perfluorocarbon liquid FC-3283 underlay (FIG. 5A-E). The suitability of FC-3283 for patchoulol extraction for this culture is evident in the clear appearance of the product peak in GC-FID chromatograms in both solvents (FIG. 5A). Patchoulol was confirmed as the appropriate product by mass fractions. FC-3283 is more dense than water and was found underneath the culture medium in contrast to the lighter dodecane, which floats on top (FIG. 5B). Both solvents enabled the algal culture to grow, with cell densities increasing throughout cultivation in standard conditions and consistent linear electron flow in both, indicating healthy cultures (FIG. 5C and 5D). Healthy green culture is also noticeable in FIG. 5B. Subsequent studies then checked the suit-ability of FC-3283 for extraction of other heterologous isoprenoid products using strains previously engineered to produce taxadiene, casbene, and 13R(+) manoyl oxide.24 Each strain generated unique products in chromatograms (FIG. 5E), as previously observed for living extraction with dodecane.²⁴ The results indicated that FC-3283 is bio-compatible and amenable to accumulating heterologous isoprenoids from our engineered algal cells.

### New Options for Two-Phase Cultivation With Underlays

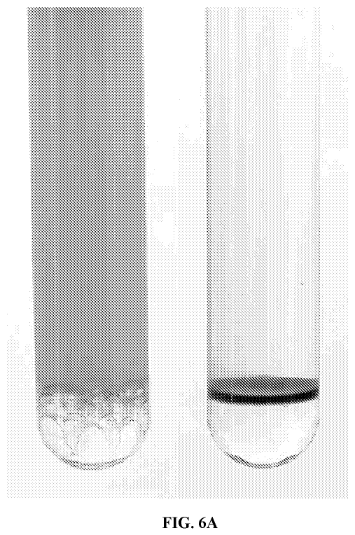

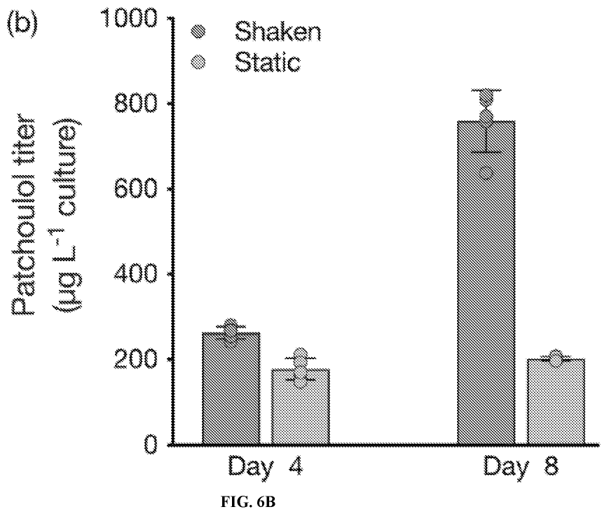

As a lower phase to growing liquid microbial culture, FCs present a new avenue for bio-process designs aimed at milking heterologous products. The present studies investigated whether the lower phase could be used as a support for microbes to grow while concomitantly enabling extraction from the biofilm. A shaken culture was compared to a static culture for their abilities to produce patchoulol (FIG. 6A-B). Both cultures grew, with the non-shaken culture accumulating a layer of cells at the medium-FC interface. After four days of cultivation, patchoulol concentration in the FC-3283 phase of the static culture was already significantly lower (177+25 μgL⁻¹ culture; mean±SD) compared to the shaken culture (263 +14 μgL⁻¹ culture) (t(8) ¼ 6.56, p<0.001). By day eight, overall patchoulol titers were four-fold lower in the static (202±5 μgL⁻¹ culture) than in the mixed culture (759±73 μgL⁻¹ culture) (t(8)=17.05, p<0.0001) FIG. 6B). It seems logical that static cultures would have lower overall production rates, as only the cells close to the interface would interact with the FC. Future concepts may involve some amount of mixing and settling, or agitation of the FC liquid below the culture to increase this interaction. The ability to grow organisms at the solvent interface could enable the cultivation of mat-forming organisms and the extraction of their natural products. For example, the members of the genus Botryococcus, often found on the surface of stagnant waters, are slow-growing algae that can secrete alkadienes and alkatrienes or botryococene isoprenoids.³⁰⁻³² Cultures set up with larger surface area interaction between medium and FC may enable interesting process designs for milking secreted products.

### Liquid-Liquid Extraction of Isoprenoids from FC-3283

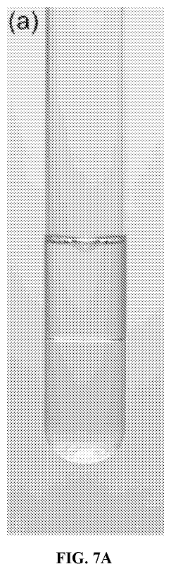

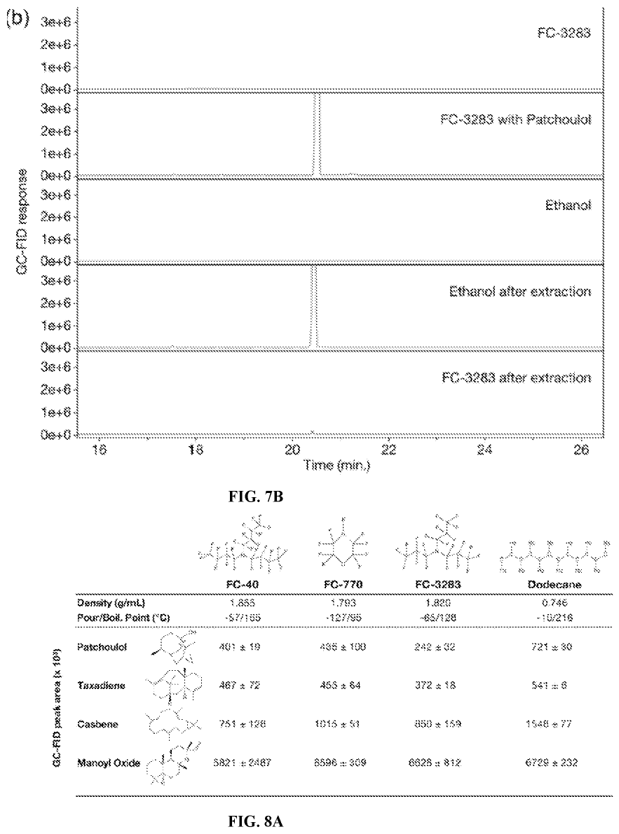

Once accumulated in a solvent, it is desired to extract and isolate the target chemicals and recycle the solvent for further use. With dodecane, silica-based solid phase extraction (SPE) allows patchoulol to be isolated from the solvent due to its hydroxyl group, however, separation is more challenging with non-functionalized isoprenoids. This challenge is different for every target compound, but many sesqui-and diterpenoids have similar chemical properties to the C12 dodecane, making processes like distillation or molecular separation more challenging. Studies sought to determine how readily one could isolate target isoprenoids from FC-3283 while regenerating the FC (FIG. 7A-B). As it is immiscible with ethanol (FIG. 7A), liquid-liquid extraction was performed on FC-3283 which had accumulated patchoulol from a C. reinhardtii cultivation. Surprisingly, patchoulol partitioned readily into the ethanol phase (FIG. 7B), which phase-separated from FC-3283 after extraction. The present studies found that the process can be completed in as fast as 2 hours of incubation (data not shown), indicating the potential to obtain formulated isoprenoid product from microbial cultures in as little as two downstream process steps.

### Extraction Potential of Other Fluorinated Liquids

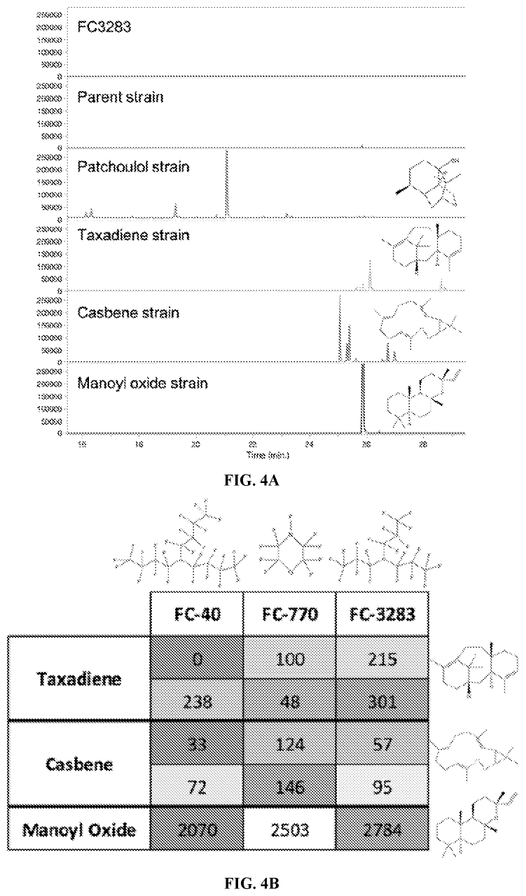

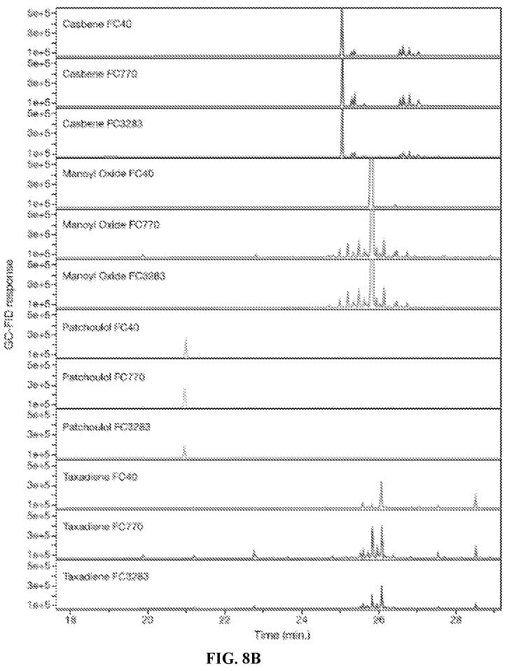

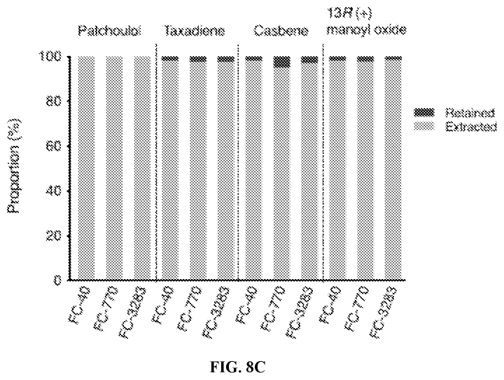

Encouraged by the results of FC-3283 microbial milking, C. reinhardtii strains producing multiple heterologous isoprenoids were cultivated with other perfluorocarbon heat-transfer liquids FC-40 and FC-770 (FIG. 4). The different FCs have unique physical properties and exhibited variable performance in extracting different isoprenoids from the algal cells (FIG. 8A). Each fluorocarbon resulted in varying extraction rates for each isoprenoid (FIG. 8A), and these behaviors will enable tailoring of bio-processes to specific product chemistries giving the operator several choices in two-phase synthetic solvents. FC-770, for example, routinely exhibited product peaks in GC chromato-grams which were absent or in reduced quantities for the other FCs (FIG. 8B). Perhaps this represents a more accurate profile of side-product isoprenoids produced by these strains. Indeed, chromatograms of FCs contain much fewer background peaks and contaminants compared to n-dodecane samples which also makes characterization more straightforward (FIG. 5A). The studies also showed that the liquid-liquid extraction with ethanol was highly effective for all analyzed isoprenoids, regardless of the FC compound used or the chemical nature of the isoprenoid. It was possible to recover patchoulol into ethanol at efficiencies >99%, while for other terpenoids the efficiency ranged from 95-98% (FIG. 8C), indicating a greater practical value than traditional solvents.

Compared to dodecane, most FCs were less efficient in isoprenoid extraction from the algal cells in batch culture (FIG. 8A). This can likely be an acceptable compromise in bio-process designs when considering the efficiencies of liquid-liquid extractability of the isoprenoid products from the FCs (FIG. 7A-B). Cultures with FC-3283 overlays also exhibited higher linear electron flow rates than those with dodecane throughout batch cultivation (FIG. 5D), indicating FC-3283 is less aggressive as a two-phase culture system than dodecane. It could be speculated that higher efficiencies of extraction with dodecane are due to microbial lysis rather than efficient partitioning, although this aspect was not investigated here.

### CONCLUSIONS

The present study is evidence that commercially available heat-transfer solvents such as perfluorocarbons and solvent which are liquid at biologically relevant temperatures can be used as bio-compatible liquids for microbial cell milking of heterologous isoprenoids. The FCs tested here showed capacities for easier handling, consistent product accumulation, and ease of product isolation compared to petroleum-based solvent counterparts. As dense liquids, FCs form an underlay with microbial cultures, which opens new avenues for bio-process designs, especially for slow-growing or mat-forming microbes. The use of FC liquids in microbial metabolite milking permits inert, room temperature extraction conditions and likely provides a route to well-preserved natural product extraction from microbial cells. Here, the present studies used engineered microalgae as a model organism, however, these results could have applicability in the synthetic biology and metabolic engineering fields for a broad spectrum of microbes.

### REFERENCES

1 Daletos, et al., Trends Biotechnol., 2020, 38, 811-822.
2 Heider, et al., BMC Microbiol., 2012, 12.
3 Lauersen, Planta, 2019, 249, 155-180.
4 Navale, et al., Appl. Microbiol. Biotechnol., 2021, 105, 457-475.
5 Wolf, et al., Essays Biochem., 2021, 65, 197-212.
6 Zhan, Y et al., Front. Plant Sci., 2014, 5, 636.
7 Wichmann, et al., Curr. Opin. Biotechnol., 2020, 61, 28-37.
8 Gershenzon et al., Nat. Chem. Biol., 2007, 3, 408-414.
9 Kirby et al. Rev. Plant Biol., 2009, 60, 335-355.
10 Jensen, et al., Nat. Commun., 2021, 12, 2260.
11 Wlodarczyk, et al., Metab. Eng., 2016, 33, 1-11.
12 Yamada, et al., Proc. Natl. Acad. Sci. U. S. A., 2015, 112, 857-862
13 Wei, et al.,. Chen, Sci. Rep., 2020, 10, 14944.
14 Beran, et al., Proc. Natl. Acad. Sci. U. S. A., 2016, 113, 2922-2927.
15 Gilg, et al., Naturwissenschaften, 2009, 96, 731-735.
16 Li, et al., Nat. Prod. Rep., 2020, 37, 80-99.
17 Peralta-Yahya, et al., Nat. Commun., 2011, 2, 483.
18 Beekwilder, et al., Plant Biotechnol. J., 2014, 12, 174-182.
19 W. Liu, X. Xu, R. Zhang, T. Cheng, Y. Cao, X. Li, J. Guo, H. Liu and M. Xian, Biotechnol. Biofuels, 2016, 9, 131.
20 I. S. Yunus and P. R. Jones, Metab. Eng., 2018, 49, 59-68.
21 M. Choi and K. Cho, Int. J. Heat Mass Transfer, 2000, 43, 209-218.
22 Jouhara et al., Appl. Therm. Eng., 2010, 30, 201-211.
23 Lauersen, et al., Metab. Eng., 2016, 38, 331-343.
24 Lauersen, et al., Metab. Eng., 2018,49, 116-127.
25 Neupert, et al., Plant J., 2009, 57, 1140-1150.
26 Gorman and R. Levine, Proc. Natl. Acad. Sci. U. S. A., 1965, 54, 1665.
27 Genty, et al., Biochim. Biophys. Acta, 1989, 990, 87-92.
28 Baker, Annu. Rev. Plant Biol., 2008, 59, 89-113.
29 Yu, et al., Algal Res., 2021, 56.
30 Eroglu et al., Bioresour. Technol., 2010, 101, 2359-2366.
31 Templier, et al., Phytochemistry, 1991, 30, 2209-2215.
32 Lozoya-Gloria, et al., in Microalgae: From Physiology to Application, ed. M. V'itov'a, IntechOpen, London, UK, 2019, pp. 123-144.
33 Hasi, et al., Appl. Opt., 2008, 47, 1010-1014.
34 Hasi, et al., Laser Part. Beams, 2013, 31, 301-305.

Those skilled in the art will recognize or be able to ascertain using no more than routine experimentation, many equivalents to the specific embodiments of the method and compositions described herein. Such equivalents are intended to be encompassed by the following claims. The invention will be further understood in view of the following non-limiting examples.

1. A process for extracting a metabolite from a raw material containing that metabolite as a constituent part comprising the steps of (a) contacting the raw material with a first extraction composition so as to extract the metabolite of interest from the raw material into the first extraction composition; (b) separating the first extraction composition containing the extracted metabolite from the raw material and (c) contacting the composition from step (b) with a second extraction composition.

2. The process of claim 1, wherein the raw material is a microbial cell culture.

3. The process of claim 1 wherein the raw material is selected from the group consisting of a cell culture comprising bacteria, yeasts, plant cell culture and small cells filamentous microbes, and algae.

4. The process of claim 3, wherein the raw material is an algae cell culture.

5. The process of claim 4, wherein the culture comprises at least one strain of microalga Chlamydomonas reinhardtii

6. The process of claim 1 wherein the second extraction composition comprises ethanol, methanol or acetone

7. The process of claim 1, wherein the second extraction composition comprises Silica-based solid phase extraction (SPE).

8. The process of claim 1, wherein the first extraction composition comprises a solvent that is a substituted (with Fl, Cl, Br or I) hydrocarbon.

9. The process of claim 8 wherein the solvent is a liquid perfluorocarbon, optionally, wherein the perfluorocarbon is selected from the group consisting of FC-3283, FC 40, FC 770 and FC 3284.

10. (canceled)

11. The process of claim 1, wherein the metabolite is an isoprenoid compound.

12. The process of claim 11, wherein the isoprenoid is selected from the group consisting of patchoulol, taxadiene, casbene, or 13R(+) manoyl oxide.

13. The process of claim 1, wherein the raw material is not plant material.

14. A system for extracting metabolite from a microbial culture comprising (a) microbial cell culture vessel comprising a microbial cell population producing the metabolite, (b) one or more extraction vessels and (c) a collection vessel.

15. The system of claim 1, wherein the cell culture vessel comprises first extraction composition as an underlay to the microbial cell population.

16. The system of claim 15, further comprising a manifold.

17. The system of claim 15 or system wherein the cell culture vessel comprises droplets of the first extraction composition.

18. The system of claim 14, wherein a portion of the culture vessel containing the first extraction solvent is in fluid communication with the extraction vessel allowing the flow of the first extraction composition to, and from the microbial cell culture vessel and the extraction vessel.

19. The system of claim 14, wherein (a) the microbial cell culture is a liquid culture, a biofilm, microbial culture in a substrate such as a matrix, foam or microencapsulation; (b) wherein at least extraction vessel comprises a second extraction composition wherein the second extraction composition is a solid or a liquid; and/or (c) wherein the second extraction composition comprises ethanol, and is optionally, in fluid communication with a collection vessel allowing for fluid flow from the extraction vessel to the collection vessel.

20. (canceled)

21. (canceled)

22. The system of claim 14, wherein:
(a) the cell culture vessel comprises a gas compartment, a hydrophobic porous membrane, and/or a means for introducing gas into the cell culture vessel for gas exchange; (b) the microbial cell culture vessel comprises metabolite-producing organisms selected from the group consisting of bacteria, yeasts, filamentous microbes, and algae; (c) the first extraction composition comprises a solvent that is a substituted (with Fl, Cl, Br or I) hydrocarbon; and/or (d) wherein the metabolite is an isoprenoid compound.

23. (canceled)

24. (canceled)

25. The system of claim 14, wherein the solvent is a liquid perfluorocarbon in cell cultivation conditions; and/or wherein the perfluorocarbon is selected from the group consisting of FC-3283, FC 40, FC 770 and FC 3284.

26. (canceled)

27. (canceled)

28. (canceled)
